(19) United States
(12) Patent Application Publication
(10) Pub. No.: US 2024/0108454 A1
(43) Pub. Date: Apr. 4, 2024

(54) PATCH TO TREAT CORNEAL PERFORATION

(71) Applicant: EyeYon Medical Ltd., Nes Ziona (IL)

(72) Inventors: Ofer Daphna, Beit Elazari (IL); Nahum Ferera, Beit Elazari (IL); Dmitry Dubson, Rehovot (IL)

(73) Assignee: EyeYon Medical Ltd., Nes Ziona (IL)

(21) Appl. No.: 17/937,505

(22) Filed: Oct. 3, 2022

(51) Int. Cl. A61F 2/14; A61L 27/54

(52) U.S. Cl. CPC A61F 2/1451; A61L 27/54; A61L 2300/406; A61L 2300/412; A61L 2300/606; A61L 2430/16

(57) ABSTRACT

An ocular implant includes an endothelial patch constructed of a clear, transparent, biologically compatible material, and which has a property-modified region that has different properties than other portions of the endothelial patch.

### FIELD OF THE INVENTION

The present invention relates generally to endothelial or ocular implants, and particularly to an endothelial patch to treat corneal perforation.

### BACKGROUND OF THE INVENTION

Corneal melt is a devastating complication of end stage corneal disease. There are both immune and infectious causes. Despite the advances in corneal transplantation and rehabilitation, once the process of corneal melting has begun, it is challenging to reverse and there are few therapeutic options.

Corneal melt may occur from conditions such as infections, sterile inflammation, or surgical/chemical injury to the cornea. Corneal melting may lead to corneal perforation (“perforation” refers to any open injury, including but not limited to, perforation, tear, break, hole, etc.).

Corneal perforation can be caused by various types of infectious and noninfectious corneal disorders. Surgical and/or nonsurgical intervention is sometimes required to close the perforation, to reform the collapsed anterior chamber, and to restore visual function. In the worst scenario, irreversible angle-closure glaucoma and microbial endophthalmitis can occur, which can lead to blindness.

There are a variety of approaches for the management of corneal perforations, from nonsurgical treatments such as bandage soft contact lenses and tissue glues, to surgical modalities such as simple cornea suturing, conjunctival flaps, multilayered amniotic membrane transplantation (AMT) and corneal grafts. The choice of the treatment depends on the size and location of the perforation and status of underlying diseases.

For example, the so-called Gunderson conjunctival flap covers the entire corneal surface. A selective pedunculated (pedicle) conjunctival flap provides an alternative method to the Gunderson flap, when only partial protection is required. The pedicle conjunctival flap can be used either as a thin flap for superficial corneal ulcer, or as a thick flap for deep corneal ulcer.

Therapeutic keratoplasty uses donor corneal grafts to repair corneal perforations. A disadvantage is a relatively high rejection rate of the donor graft, due to various biological and physiological factors.

### SUMMARY

The present invention relates to an endothelial patch to treat corneal perforation, as is described more in detail hereinbelow.

The patch may be inserted into the eye and attached (coupled) to the posterior portion of the cornea in order to seal the injured area.

The size and final shape of the patch may depend on the size, shape and location of the perforated area. The patch may be sutured or attached by a biological or chemical adhesive or by natural healing. The patch may be removed after corneal healing or can be used as a permanent implant.

There is provided in accordance with a non-limiting embodiment of the present invention an ocular implant including an endothelial patch constructed of a clear, transparent, biologically compatible material, the endothelial patch including a property-modified region which has different properties than other portions of the endothelial patch.

The property-modified region may include a drug-coating or a drug-eluting patch. The drug-coating or the drug-eluting patch may release a drug to promote growth of new corneal cells, or an anti-fungal drug, an antiviral drug, or an antibacterial drug.

### BRIEF DESCRIPTION OF THE DRAWINGS

The present invention will be understood and appreciated more fully from the following detailed description taken in conjunction with the drawings in which:

FIG. 1 is a simplified perspective illustration of endothelial patch to treat corneal perforation, constructed and operative in accordance with a non-limiting embodiment of the present invention.

### DETAILED DESCRIPTION

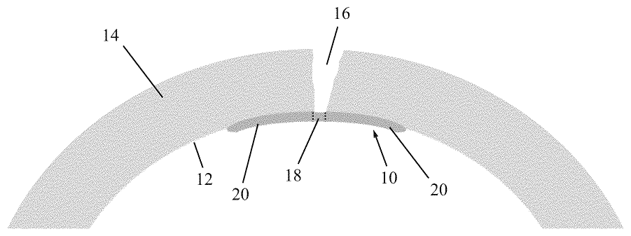

Reference is now made to FIG. 1, which illustrates an endothelial patch 10, constructed and operative in accordance with a non-limiting embodiment of the present invention.

Patch 10 is shown attached to a posterior surface 12 of a cornea 14. Patch 10 covers the posterior of a corneal perforation 16 and provides structural integrity to the posterior surface 12 of cornea 14, thereby helping the healing process until the cornea heals and presents with no corneal perforation.

Patch 10 may be flat or dome-shaped. The size and final shape of patch 10 may depend on the size, shape and location of the corneal perforation. The shape may be circular, oval, toroidal (doughnut), arch-shaped for peripheral perforation, irregularly shaped, or any other shape or size which is required.

As opposed to grafts from donor corneas, patch 10 may be constructed of a clear, transparent, biologically compatible material, such as but not limited to, polymethylmethacrylate (PMMA), silicone, silicone rubber, collagen, hyaluronic acid (including the sodium, potassium and other salts thereof), hydrogel, such as acrylic or methacrylic hydrogels, e.g., hydroxyethyl methacrylate or methacrylic acid copolymer/partially hydrolyzed poly(2-hydroxyethyl methacrylate) (known as PolyHEMA), polysulfones, thermolabile materials and other relatively hard or relatively soft and flexible biologically inert optical materials, or any combination of such materials, such as a gel encapsulated in a polymer. Patch 10 may thus be rigid, semi-rigid or foldable, for example. Some or all of patch 10 may be hydrophilic or hydrophobic.

Patch 10 may be made of a copolymer of hydroxyethyl methacrylate and methyl methacrylate, commercially available as Ci26 from Contamac Ltd., Saffron Walden, Essex, UK. Ci26 is a random, crosslinked, acrylate based copolymer consisting of poly [(methylmethacrylate)-co-(2-hydroxyethyl methacrylate)-co-(ethylene glycol dimethacrylate)], that is, it is a copolymer of methylmethacrylate (MMA) and 2-hydroxyethyl methacrylate (HEMA) and ethylene glycol dimethacrylate (EGDM). Methyl methacrylate (MMA) is a hydrophobic monomer that forms a homopolymer that does not substantially absorb water. 2-hydroxyethyl methacrylate (HEMA) is a modification of MMA, in which the non-polar pendant methyl group of MMA is replaced with a polar hydroxyethyl functional group. When HEMA is made into a homopolymer (pHEMA), it retains a hydrophobic backbone structure but the polar pendant groups allow water to be absorbed into the polymer matrix. Fully hydrated hydrogels of pHEMA typically contain up to 40% water by weight. EGDM contains two methacrylate functionalities polymerized to form cross-links between the polymer chains, MMA and HEMA, and is hydrophobic in nature. Ci26 is a blend of approximately 14% MMA, 85% HEMA, and <1% of EGDM, producing a material that can absorb water, and when fully hydrated will contain 26% water by weight. The material therefore contains a mixture of both hydrophilic and hydrophobic components.

Patch 10 may be inserted into the eye, without limitation, through a 1.8-3.0 mm incision, alternatively a 1.8-2.7 mm incision, alternatively a 1.8-2.4 mm incision, alternatively a 2.0-2.4 mm incision, and preferably a 2.2-2.4 mm incision.

Patch 10 may be attached to the posterior surface 12 of the cornea 14, such as but not limited to, by suturing, bonding with a biological or chemical adhesive or viscoelastic gel, or by natural healing. Patch 10 may be removed after corneal healing or can be used as a permanent implant.

In accordance with a non-limiting embodiment of the present invention, patch 10 may include a property-modified region 18 which has different properties than other portions 20 of patch 10. For example, the property-modified region 18 may include a drug-coating or a drug-eluting patch that releases a drug to promote growth of new corneal cells, or anti-fungal or antiviral or antibacterial drugs, or any combination thereof. As another example, the property-modified region 18 may have a refractive index different from other portions 20 of patch 10. The refractive index of property-modified region 18 may compensate for the change in refractive properties of the cornea 14 at the perforation 16.

Alternatively, one or more of portions 20 may be the property-modified region instead of region 18. The property-modified region or regions may be located on any portion of patch 10 according to a desired treatment plan.

What is claimed is:

1. An ocular implant comprising:
an endothelial patch constructed of a clear, transparent, biologically compatible material, said endothelial patch comprising a property-modified region which has different properties than other portions of said endothelial patch.

2. The ocular implant according to claim 1, wherein said property-modified region comprises a drug-coating or a drug-eluting patch.

3. The ocular implant according to claim 2, wherein said drug-coating or said drug-eluting patch releases a drug to promote growth of new corneal cells.

4. The ocular implant according to claim 2, wherein said drug-coating or said drug-eluting patch releases an anti-fungal drug.

5. The ocular implant according to claim 2, wherein said drug-coating or said drug-eluting patch releases an antiviral drug.

6. The ocular implant according to claim 2, wherein said drug-coating or said drug-eluting patch releases an antibacterial drug.

7. The ocular implant according to claim 1, wherein said property-modified region has a refractive index different from the other portions of said endothelial patch.

8. The ocular implant according to claim 1, wherein said clear, transparent, biologically compatible material is hydrophilic.

9. The ocular implant according to claim 1, wherein said clear, transparent, biologically compatible material has both hydrophilic and hydrophobic properties.

10. The ocular implant according to claim 1, wherein said clear, transparent, biologically compatible material comprises a copolymer of hydroxyethyl methacrylate and methyl methacrylate.
